(19) United States
(12) Patent Application Publication
(10) Pub. No.: US 2017/0007776 A1
(43) Pub. Date: Jan. 12, 2017

(54) CANNULA, AN INJECTION OR INFUSION DEVICE AND METHODS OF USING THE CANNULA OR THE INJECTION OR INFUSION DEVICE

(71) Applicants: BIONTECH AG, Mainz (DE); TRON - Translationale Onkologie an der Universitätsmedizin der Johannes Gutenbert-Univer, Mainz (DE)

(72) Inventor: Ugur Sahin, Mainz (DE)

(21) Appl. No.: 15/115,384

(22) PCT Filed: Feb. 5, 2014
(86) PCT No.: PCT/EP2014/000306

§ 371 Date: Jul. 29, 2016

(51) Int. Cl. A61M 5/32; A61M 5/315

(52) U.S. Cl. CPC A61M 5/3291; A61M 5/3286; A61M 5/315

(57) ABSTRACT

The present invention provides a cannula (300) with a distal end (311) and a proximal end (312), comprising a sidewall (301) defining a lumen (302) for receiving a fluid (800) from a feeding device (400, 500), wherein the sidewall (301) comprises a plurality of openings (303) for supplying the fluid (800) into a target tissue (700) of a patient. Each opening (304) is in fluid communication with the lumen (302) of the cannula (300). Furthermore, an injection or infusion device (100, 200) is provided, comprising said cannula (300) and a feeding device (400, 500) for supplying the fluid (800) to the cannula (300), wherein the cannula (300) is attached or is configured to be attachable to the feeding device (400, 500). Corresponding methods are also provided, using said cannula and/or said injection or infusion device (100, 200).

### RELATED APPLICATIONS

This application is a national stage application submitted under 35. U.S.C. 371 of International Patent Application No. PCT/EP2014/00306, filed on Feb. 5, 2014, the contents of which are hereby enclosed in their entirety.

### TECHNICAL FIELD

The present invention relates to the therapeutic administration of RNA to a patient.

### BACKGROUND

RNA plays a common and vital role in all living organisms and is a critical element in protein production. RNA-based therapeutics are believed to attack diseases or disorders in a fundamental manner. RNA may be directly injected into the patient and expressed in cells of the patient.

The use of RNA provides an attractive alternative to circumvent the potential risks of DNA based therapeutics such as DNA based vaccines. The advantages of using RNA as a kind of reversible gene therapy include transient expression and a non-transforming character. RNA does not need to enter the nucleus in order to be expressed and moreover cannot integrate into the host genome, thereby eliminating the risk of oncogenesis. Furthermore, transfection rates attainable with RNA are relatively high and the amounts of protein achieved correspond to those in physiological expression.

Several successful approaches to stabilize RNA by various modifications in order to achieve higher and prolonged expression of transferred RNA have been reported. Furthermore, success has been achieved in issues related to cytotoxicity of RNA. However, it has been found that intramuscular administration of RNA using common injection devices results in the RNA only being taken up and expressed in a limited number of cells. This is due to the fact that the RNA is dispensed only at the opening of a cannula where a single reservoir is formed from which the RNA can only be taken up and expressed by a limited number of cells surrounding the reservoir.

The aim of the present invention is to provide a device suitable for administering RNA to a patient. Such device should result in the RNA administered not only being taken up by cells of the patient surrounding the opening of a cannula.

### SUMMARY

In a first aspect, the present invention relates to a cannula with a distal end and a proximal end. The cannula comprises a sidewall defining a lumen for receiving a fluid from a feeding device. The sidewall comprises a plurality of openings for supplying or applying the fluid into a target tissue of a patient, e.g. into a muscle, wherein each opening is in fluid communication with the lumen of the cannula.

Thus, the cannula forms a tube, in particular a cylindrical tube, wherein the sidewall or the lateral surface comprises the plurality of openings. The openings are configured so that the fluid is applicable or may be administered to or into the desired target of the human or animal patient.

In one embodiment, the cannula is an injection cannula (also hypodermic needle or cannula) or an infusion cannula for treating a patient's body in the medical or veterinary field. The cannula may be provided for the immediate removal after administering the fluid into the target tissue or as an indwelling cannula or a butterfly cannula which is configured for remaining in the target tissue for a determined period of time.

In one embodiment, the cannula comprises a (target) tissue penetrating tip on the distal end. Therefore, the target tissue may be reached and treated with the cannula, thereby avoiding the use of additional components.

However, it might be necessary to introduce the cannula into an already existing opening, e.g. a channel, wherein attention has to be paid not to hurt, e.g. penetrate, the surrounding tissue.

Therefore, in one embodiment, a buttoned cannula is provided for treating a patient's body, which comprises a button on the distal end, that is a blunted or button tip. The blunted tip may comprise a button or knob which covers the tip to protect the tissue. The button may be formed as an integral component with the cannula or may be provided as an additional element which is attachable to the cannula.

In one embodiment, the plurality of openings is evenly distributed on the sidewall and/or entirely covers the sidewall. The release or leakage or discharge and the distribution of the fluid within the target tissue depends on the number and the size of the openings in the sidewall or lateral surface of the cannula. It can be expected that the number of cells of the target tissue or organ which is contacted with the fluid increases with an increasing number of openings in the cannula and thus, with a more evenly distribution of the fluid in the target tissue or organ. Furthermore, the higher the number of cells of the target tissue or organ which is contacted with the fluid, the higher the number of cells which will take up and preferably express RNA contained in the fluid. Depending on the substance to be applied to the target tissue, different cannulas may be used. Thus, it is possible to control the discharge of the fluid into the tissue, e.g. into the corresponding muscle.

In one embodiment, the plurality of openings forms at least one row which extends from the proximal end of the cannula to the distal end. That is, the row runs along a direction of extension or along a longitudinal axis of the cannula from the proximal end to the distal end. A plurality of rows may be provided, which extend off-set to one another or which extend such that adjacent openings are provided at the same height on the sidewall, with respect to the longitudinal axis. In the first case the available space on the sidewall may be optimally used, and thus, more openings may be provided compared to the latter case. In one embodiment the plurality of rows envelope the entire sidewall, and therefore, two, three, four, five or more rows are provided.

In an alternative embodiment, the one or more rows extend in a helical manner from the proximal end of the cannula to the distal end. Preferably, the helical rows are formed and/or arranged such that the openings along the cannula do not “overlap” or do not completely “overlap”, i.e. the openings do not lie upon each other or do not completely lie upon each other. In other words, each opening is arranged on an axis parallel to the longitudinal axis of the cannula, wherein each longitudinal axis only comprises one opening.

In one embodiment, the openings comprise circular, oval and/or a slit-shaped structure. Also a grid-shaped structure may be provided for forming the openings.

In one embodiment, the target tissue penetrating tip and/or the button tip comprise a tip opening.

In one embodiment, the cannula is attached or connected to or mounted to, on or onto or is configured to be attachable or connectable to or mountable to, on or onto the feeding device of an injection or infusion device. The feeding device is provided for supplying the fluid to the cannula. The cannula and the feeding device may be formed as an integral unit or in one piece, or may be provided as two or more single components, wherein the cannula may be attached to the feeding device by means of a connection device or connecting device. The connection device may be provided for example as a press-fit or twist-on fitting, for example a screw coupling such as a Luer lock.

In one embodiment, the cannula comprises or is embedded in a connection element or connecting element which forms a part of the connection device. The connection element may be preferably provided as a funnel-shaped, that is a female element, which allows for attaching the cannula to the feeding device. The feeding device comprises a corresponding element, e.g. a male element, for receiving the female element and thus the cannula.

The connection element may be made of plastics or aluminium. Female and male element form the connection device of the injection or infusion device.

In case of an infusion device, the connection element may comprise a wing element, wherein the connection element and the wing element are preferably provided in one piece. In this case, the injection or infusion device is an indwelling catheter device or a butterfly device and the cannula is an indwelling cannula or butterfly cannula. The wing element is used for providing a stable position of the cannula within the tissue and serves as a supporting surface.

In one embodiment, the cannula comprises a length in the range of 10 to 120 mm, preferably in the range of 10 to 50 mm, preferably in the range of 10 to 30 mm, such as in the range of 12 to 13 mm or 22 to 26 mm, preferably in the range of 22.2 to 25.4 mm. In particular embodiments, the cannula comprises a length of 20, 23, 24, 25, 30, 40, 50, 60, 70 or 80 mm.

In one embodiment, the cannula comprises an outer diameter in the range of 0.5 to 1.2 mm, preferably of 0.6, 0.9 or 1.0 mm, e.g. for oily emulsions, and preferably of 0.5, 0.4, 0.3, 0.2 or 0.1 mm for other substances.

Typical known cannulas are characterized as 0.45×12, 0.5×16, 0.7×30, 0.8×40, 0.9×40, 1.1×40, 1.2×40, 0.6×60, 0.9×70 cannulas, wherein the first numeral indicates the outer diameter of the cannula in mm and the second numeral indicates the length of the cannula in mm (see also Gauge system).

In one embodiment, the material of the cannula is of stainless steel, e.g. a steel tube, of polytetrafluoroethylene, of polyurethane, of polyamide or of silicone. Flexible cannulas like polytetrafluoroethylene cannulas may be configured to receive a guiding mandrel or guiding needle. The guiding element allows for guiding or directing the cannula to the desired region, that is the target tissue, and may be removed after positioning the cannula in the target tissue. Thus, the cannula is configured for remaining in the target tissue for a predetermined period of time. The guiding mandrel preferably comprises a tissue penetrating tip. Otherwise, the cannula has to be introduced into the target tissue via a preformed or natural opening or channel or a similar access.

In one embodiment, the cannula is coated with a hydrogel and/or a drug for increasing the slide characteristics of the cannula's material and/or for providing an antiseptic surface of the cannula. Also a blood-repellent surface of the cannula may allow for an easier access to the target tissue and for an easier treatment of the patient.

In one embodiment, the cannula is formed as a disposable component. Disposable needles or cannulas are far more common in medicine. However, also reusable cannulas are applicable.

The present invention is also applicable to bent or curved cannulas which may be used in the medical or veterinary field.

In a further aspect, the present invention relates to an injection or infusion device. The device comprises a cannula as described above and a feeding device for supplying the fluid to the cannula. The cannula is attached or connected to or mounted to, on or onto or is configured to be attachable or connectable to or mountable to, on or onto the feeding device.

In one embodiment, the feeding device comprises a tube-shaped or cylindrical element or a syringe barrel, comprising a distal end and a proximal end, the tube-shaped or cylindrical element or syringe barrel for receiving the fluid to be supplied into the target tissue or organ of a patient. Furthermore, the device comprises a piston element sealingly slidable within the tube-shaped or cylindrical element or syringe barrel by means of a piston rod. The piston rod is connected with the piston element for moving the piston element relative to the tube-shaped or cylindrical element or syringe barrel. In this case, the injection or infusion device is for example provided as a syringe device, which is used to administer a fluid into the target tissue or organ and which is configured to be immediately removed from the target tissue or organ after the application of the fluid.

In one embodiment, the feeding device comprises a reservoir which contains the fluid to be supplied into the target tissue or organ of a patient, and a connection tube or hose for connecting the reservoir with the cannula. In this case, the injection or infusion device is for example provided as an indwelling catheter device or a butterfly device and the cannula is provided as an indwelling cannula or butterfly cannula.

In one embodiment, the cannula is attached or is configured to be attachable to the feeding device by means of a connection device. The connection device includes at least two connection elements, one provided at the cannula, a further one provided at the feeding device. A syringe device may comprise a hub or collar on the distal end of the tube-shaped element, for example a male element, and a corresponding element, for example a female element, at the cannula. A similar arrangement may be provided in respect of an indwelling catheter device.

The connection device, preferably the connection element at the cannula, may comprise at least one additional inlet and/or for example a three way cock for applying additional fluids, substances or drugs to the tissue target.

In one embodiment, the feeding device and the cannula are formed as an integral component or in one piece. However, single components which are connectable via the connection device may also be provided.

According to the invention, the fluid is preferably a fluid comprising RNA such as a liquid pharmaceutical composition comprising RNA. In a further aspect; the present invention relates to an injection or infusion device as described above wherein the feeding device is charged with the fluid to be administered. Such injection or infusion device may be intended for immediate use by the patient or health care personnel since it does not require that the injection or infusion device is loaded with the fluid prior to its use.

The cannula or injection or infusion device described herein may be provided in a packaging, preferably an aseptic packaging such as a plastic container or plastic bag. Alternatively or additionally, the cannula or injection or infusion device may be part of a kit which may contain instructions for using the cannula or injection or infusion device, e.g. instructions for using the cannula or injection or infusion device in the methods of the invention.

Preferably, the RNA contained in the fluid once administered to a target organ or tissue, preferably by intramuscular injection, using the cannula or injection or infusion device described above is taken up and expressed by cells of the target organ or tissue resulting in the production of peptide(s) and/or protein(s) encoded by the RNA.

Thus, in a further aspect, the present invention relates to a method for delivering RNA to cells of a subject, comprising administering to the subject a fluid comprising the RNA using the cannula described above or the injection or infusion device described above.

In a further aspect, the present invention relates to a method for expressing RNA in cells of a target organ or tissue comprising introducing into the target organ or tissue a fluid comprising the RNA using the cannula described above or the injection or infusion device described above. Preferably, the RNA once introduced into the target organ or tissue is taken up by cells of the target organ or tissue.

In a further aspect, the present invention relates to a method of treating or preventing a disease in a patient comprising administering to the patient a fluid comprising RNA using the cannula described above or the injection or infusion device described above. Preferably, the RNA encodes a peptide or protein having a therapeutically beneficial effect on said disease.

### BRIEF DESCRIPTION OF THE DRAWINGS

FIG. 1 shows a schematic side view of an injection device according to an embodiment of the present invention; merely the piston element with the piston rod, which in fact are merely visible in part, are completed by means of dashed lines.

FIG. 2 shows a part of a cannula according to an embodiment of the present invention, positioned in a target tissue of a patient.

FIG. 3 shows a part of a conventional cannula, positioned in a target tissue of a patient.

FIG. 4 shows a schematic side view of an infusion device according to an embodiment of the present invention.

FIG. 5 shows a perspective view of a part of the cannula shown in FIG. 2.

FIG. 6 shows a perspective view of a part of a cannula according to a further embodiment of the present invention.

FIG. 7 shows a perspective view of a part of a cannula according to a further embodiment of the present invention.

FIG. 8 shows a perspective view of a part of a cannula according to a further embodiment of the present invention.

FIGS. 9A-9B show Beta-Galactosidase expression after four single injections of lacZ-GFP-luc Replicon RNA in musculus tibialis posterior of balb/c mouse. FIG. 9A shows an overview of a 12 μm cryo cross-section (5× magnification). FIG. 9B shows two single beta-Gal positive cells (20× magnification).

### DETAILED DESCRIPTION OF PREFERRED EMBODIMENTS

The present invention will now be further described by defining different aspects of the invention generally outlined above in more detail. Each aspect so defined may be combined with any other aspect or aspects unless clearly indicated to the contrary. In particular, any feature indicated as being preferred or advantageous may be combined with any other feature or features indicated as being preferred or advantageous.

Although the present invention is described in detail below, it is to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

A nucleic acid is according to the invention preferably deoxyribonucleic acid (DNA) or ribonucleic acid (RNA) such as in vitro transcribed RNA (IVT RNA). Nucleic acids include according to the invention recombinantly produced and chemically synthesized molecules. According to the invention, a nucleic acid may be present as a single-stranded or double-stranded and linear or covalently circularly closed molecule. A nucleic acid can, according to the invention, be isolated. The term “isolated nucleic acid” means, according to the invention, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e. transfection of, cells, in particular, in the form of RNA which can be prepared by in vitro transcription from a DNA template. The RNA can moreover be modified before application by stabilizing sequences, capping, and polyadenylation.

In the context of the present invention, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. The term “RNA” comprises double-stranded RNA, single-stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA. In one embodiment of the invention, RNA is not chemically modified. In one embodiment of the invention, RNA only comprises standard nucleotides, such as naturally occurring nucleotides.

According to the present invention, the term “RNA” includes and preferably relates to “mRNA”. The term “mRNA” means “messenger-RNA” and relates to a “transcript” which encodes a peptide or protein and may be generated by using a DNA template. Typically, mRNA comprises a 5′-UTR, a protein coding region, and a 3′-UTR. mRNA only possesses limited half-life in cells and in vitro. In the context of the present invention, mRNA may be generated by in vitro transcription from a DNA template. The in vitro transcription methodology is known to the skilled person. For example, there is a variety of in vitro transcription kits commercially available.

In one embodiment of the present invention, RNA is self-replicating RNA, such as single stranded self-replicating RNA. In one embodiment, the self-replicating RNA is single stranded RNA of positive sense. In one embodiment, the self-replicating RNA is viral RNA or RNA derived from viral RNA. In one embodiment, the self-replicating RNA is alphaviral genomic RNA or is derived from alphaviral genomic RNA. In one embodiment, the self-replicating RNA is a viral gene expression vector. In one embodiment, the virus is Semliki forest virus. In one embodiment, the self-replicating RNA contains one or more transgenes which in one embodiment, if the RNA is viral RNA, may partially or completely replace viral sequences such as viral sequences encoding structural proteins. In one embodiment, the self-replicating RNA is introduced into a cell in the form of in vitro transcribed RNA.

According to the invention, the stability and translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448 incorporated herein by reference. In order to increase expression of the RNA used according to the present invention, it may be modified within the coding region, i.e. the sequence encoding the expressed peptide or protein, preferably without altering the sequence of the expressed peptide or protein, so as to increase the GC-content to increase mRNA stability and to perform a codon optimization and, thus, enhance translation in cells.

The term “modification” in the context of the RNA used in the present invention includes any modification of an RNA which is not naturally present in said RNA.

In one embodiment of the invention, the RNA used according to the invention does not have uncapped 5′-triphosphates. Removal of such uncapped 5′-triphosphates can be achieved by treating RNA with a phosphatase.

The RNA according to the invention may have modified ribonucleotides in order to increase its stability and/or decrease cytotoxicity. For example, in one embodiment, in the RNA used according to the invention 5-methylcytidine is substituted partially or completely, preferably completely, for cytidine. Alternatively or additionally, in one embodiment, in the RNA used according to the invention pseudouridine is substituted partially or completely, preferably completely, for uridine.

In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap (m7G). In the context of the present invention, the term “5′-cap” includes a 5′-cap analog that resembles the RNA cap structure and is modified to possess the ability to stabilize RNA and/or enhance translation of RNA if attached thereto, preferably in vivo and/or in a cell.

Providing RNA with a 5′-cap or 5′-cap analog may be achieved by in vitro transcription of a DNA template in presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

The RNA may comprise further modifications. For example, a further modification of the RNA used in the present invention may be an extension or truncation of the naturally occurring poly(A) tail or an alteration of the 5′- or 3′-untranslated regions (UTR) such as introduction of a UTR which is not related to the coding region of said RNA, for example, the exchange of the existing 3′-UTR with or the insertion of one or more, preferably two copies of a 3′-UTR derived from a globin gene, such as alpha2-globin, alpha1-globin, beta-globin, preferably beta-globin, more preferably human beta-globin.

RNA having an unmasked poly-A sequence is translated more efficiently than RNA having a masked poly-A sequence. The term “poly(A) tail” or “poly-A sequence” relates to a sequence of adenyl (A) residues which typically is located on the 3′-end of a RNA molecule and “unmasked poly-A sequence” means that the poly-A sequence at the 3′ end of an RNA molecule ends with an A of the poly-A sequence and is not followed by nucleotides other than A located at the 3′ end, i.e. downstream, of the poly-A sequence. Furthermore, a long poly-A sequence of about 120 base pairs results in an optimal transcript stability and translation efficiency of RNA.

Therefore, in order to increase stability and/or expression of the RNA used according to the present invention, it may be modified so as to be present in conjunction with a poly-A sequence, preferably having a length of 10 to 500, more preferably 30 to 300, even more preferably 65 to 200 and especially 100 to 150 adenosine residues. In an especially preferred embodiment the poly-A sequence has a length of approximately 120 adenosine residues. To further increase stability and/or expression of the RNA used according to the invention, the poly-A sequence can be unmasked.

In addition, incorporation of a 3′-non translated region (UTR) into the 3′-non translated region of an RNA molecule can result in an enhancement in translation efficiency. A synergistic effect may be achieved by incorporating two or more of such 3′-non translated regions. The 3′-non translated regions may be autologous or heterologous to the RNA into which they are introduced. In one particular embodiment the 3′-non translated region is derived from the human β-globin gene.

A combination of the above described modifications, i.e. incorporation of a poly-A sequence, unmasking of a poly-A sequence and incorporation of one or more 3′-non translated regions, has a synergistic influence on the stability of RNA and increase in translation efficiency.

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA. The half-life of RNA may influence the “duration of expression” of the RNA. It can be expected that RNA having a long half-life will be expressed for an extended time period.

The term “recombinant” in the context of the present invention means “made through genetic engineering”. Preferably, a “recombinant entity” such as recombinant RNA in the context of the present invention is not occurring naturally, and preferably is a result of a combination of entities such as nucleic acid sequences which are not combined in nature. For example, recombinant RNA in the context of the present invention may contain several nucleic acid sequences derived from different nucleic acids fused together.

The term “naturally occurring” as used herein refers to the fact that an object can be found in nature. For example, a protein or nucleic acid that is present in an organism (including viruses) and can be isolated from a source in nature and which has not been intentionally modified by man in the laboratory is naturally occurring.

The term “expression” is used according to the invention in its most general meaning and comprises the production of RNA and/or peptides or proteins, e.g. by transcription and/or translation. With respect to RNA, the term “expression” or “translation” relates in particular to the production of peptides or proteins. It also comprises partial expression of nucleic acids. Moreover, expression can be transient or stable.

According to the invention, terms such as “RNA expression”, “expressing RNA”, or “expression of RNA” relate to the production of peptide or protein encoded by the RNA. Preferably, such terms relate to the translation of RNA so as to express, i.e. produce peptide or protein encoded by the RNA.

In the context of the present invention, the term “transcription” relates to a process, wherein the genetic code in a DNA sequence is transcribed into RNA. Subsequently, the RNA may be translated into protein. According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, appropriate DNA templates such as transcription vectors are applied for the generation of transcripts. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA. According to the present invention, the RNA used in the present invention preferably is in vitro transcribed RNA (IVT-RNA).

The term “translation” according to the invention relates to the process in the ribosomes of a cell by which a strand of messenger RNA directs the assembly of a sequence of amino acids to make a peptide or protein.

Expression control sequences or regulatory sequences, which according to the invention may be linked functionally with a nucleic acid, can be homologous or heterologous with respect to the nucleic acid. A coding sequence and a regulatory sequence are linked together “functionally” if they are bound together covalently, so that the transcription or translation of the coding sequence is under the control or under the influence of the regulatory sequence. If the coding sequence is to be translated into a functional protein, with functional linkage of a regulatory sequence with the coding sequence, induction of the regulatory sequence leads to a transcription of the coding sequence, without causing a reading frame shift in the coding sequence or inability of the coding sequence to be translated into the desired protein or peptide.

The term “expression control sequence” or “regulatory sequence” comprises, according to the invention, promoters, ribosome-binding sequences and other control elements, which control the transcription of a nucleic acid or the translation of the derived RNA. In certain embodiments of the invention, the regulatory sequences can be controlled. The precise structure of regulatory sequences can vary depending on the species or depending on the cell type, but generally comprises 5′-untranscribed and 5′- and 3′-untranslated sequences, which are involved in the initiation of transcription or translation, such as TATA-box, capping-sequence, CAAT-sequence and the like. In particular, 5′-untranscribed regulatory sequences comprise a promoter region that includes a promoter sequence for transcriptional control of the functionally bound gene. Regulatory sequences can also comprise enhancer sequences or upstream activator sequences.

According to the invention, RNA may be RNA of singular molecular species, preferably encoding only a single peptide or protein. In particular embodiments, the RNA according to the invention comprises a population of different RNA molecules, e.g. a mixture of different RNA molecules optionally encoding different peptides and/or proteins, whole-cell RNA, an RNA library, or a portion of thereof, e.g. a library of RNA molecules expressed in a particular cell type, such as undifferentiated cells, in particular stem cells such as embryonic stem cells, or a fraction of the library of RNA molecules such as RNA with enriched expression in undifferentiated cells, in particular stem cells such as embryonic stem cells relative to differentiated cells. Thus, according to the invention, the term “RNA” may include a mixture of RNA molecules, whole-cell RNA or a fraction thereof, which may be obtained by a process comprising the isolation of RNA from cells and/or by recombinant means, in particular by in vitro transcription.

Preferably, according to the invention, the RNA taken up or introduced into a cell is expressed in said cell, i.e. peptide(s) and/or protein(s) encoded by the RNA are produced in the cell. The cell may express the encoded peptide or protein intracellularly (e.g. in the cytoplasm and/or in the nucleus), may secrete the encoded peptide or protein, or may express it on the surface.

According to the invention, the term “RNA encoding” means that the RNA, if present in the appropriate environment, preferably within a cell, can direct the assembly of amino acids to produce the protein or peptide is encodes during the process of translation. Preferably, RNA according to the invention is able to interact with the cellular translation machinery allowing translation of the protein or peptide.

Terms such as “transferring”, “introducing” or “transfecting” are used interchangeably herein and relate to the incorporation or uptake of nucleic acids, in particular exogenous or heterologous nucleic acids, in particular RNA, into a cell. Said terms also include the repetitive introduction of nucleic acids, in particular RNA, into a cell, wherein repetitive mean more than once, e.g. two times or more, three times or more, four times or more, five times or more, six times or more, seven times or more, eight times or more. The time interval between said repetitive introductions of nucleic acids may be 3 days or less, 2 days or less, 24 hours or less or even lower.

Preferably, according to the present invention, a cell into which RNA is introduced forms part of an organ or tissue, such as muscle, into which the RNA is injected using the cannula or injection or infusion device described herein.

According to the present invention, the administration of RNA is either achieved as naked RNA or in combination with an administration reagent. Preferably, administration of RNA is in the form of naked RNA. In one embodiment, the RNA is administered in combination with stabilizing substances such as RNase inhibitors.

The term “cell” preferably relates to an intact cell, i.e. a cell with an intact membrane that has not released its normal intracellular components such as enzymes, organelles, or genetic material. An intact cell preferably is a viable cell, i.e. a living cell capable of carrying out its normal metabolic functions. Preferably said term relates according to the invention to any cell which can be transformed or transfected with an exogenous nucleic acid. In one embodiment, the cell is a somatic cell such as a muscle cell. Preferably, the cell is a human cell.

According to the invention, RNA may be associated with any carriers with which RNA can be associated, e.g. by forming complexes with the RNA or forming vesicles in which the RNA is enclosed or encapsulated, preferably resulting in increased stability of the RNA compared to naked RNA. Carriers useful according to the invention include, for example, lipid-containing carriers such as cationic lipids, liposomes, in particular cationic liposomes, and micelles.

Cationic lipids may form complexes with negatively charged nucleic acids. Any cationic lipid may be used according to the invention.

In one embodiment of the present invention, the RNA is associated with at least one agent having a stabilizing effect on the RNA. In one embodiment, the stabilizing effect comprises protection from RNA degradation. In one embodiment, the at least one agent forms a complex with and/or encloses said RNA. In one embodiment, the at least one agent comprises a cationic compound, preferably a polycationic compound. In one embodiment, the at least one agent comprises at least one agent selected from the group consisting of an RNA-complexing lipid, an RNA complexing polymer and an RNA-complexing peptide or protein. In one embodiment, the at least one agent comprises at least one agent selected from the group consisting polyethyleneimine, protamine, a poly-L-lysine, a poly-L-arginine or a histone. In one embodiment, the at least one agent is comprised in a vesicle enclosing said RNA, wherein the vesicle preferably is a multilamellar vesicle, an unilamellar vesicle, or a mixture thereof. In one embodiment, the vesicle is a liposome, preferably a cationic liposome. In one embodiment, the liposome comprises a phospholipid such as phosphatidylcholine and/or a sterol such as cholesterol.

According to the present invention, the term “peptide” comprises oligo- and polypeptides and refers to substances comprising two or more, preferably 3 or more, preferably 4 or more, preferably 6 or more, preferably 8 or more, preferably 10 or more, preferably 13 or more, preferably 16 more, preferably 21 or more and up to preferably 8, 10, 20, 30, 40 or 50, in particular 100 amino acids joined covalently by peptide bonds. The term “protein” refers to large peptides, preferably to peptides with more than 100 amino acid residues, but in general the terms “peptides” and “proteins” are synonyms and are used interchangeably herein.

The term “peptide” or “protein” also includes “variants” of naturally occurring peptides or proteins.

For the purposes of the present invention, “variants” of an amino acid sequence comprise amino acid insertion variants, amino acid addition variants, amino acid deletion variants and/or amino acid substitution variants.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more amino acids.

Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants.

Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids.

According to the present invention, the RNA preferably is coding RNA, i.e. RNA encoding a peptide or protein. According to the invention, RNA preferably comprises or consists of pharmaceutically active RNA. In preferred embodiments, the RNA encodes a peptide or protein which is of therapeutic value, i.e. which has a therapeutically beneficial effect in a patient. For example, said RNA may be RNA encoding and expressing an antigen or an immunologically active compound (which does not encode an antigen). Alternatively, the RNA can be non-coding RNA such as antisense-RNA, micro RNA (miRNA) or siRNA.

A “pharmaceutically active RNA” is a RNA that encodes a pharmaceutically active peptide or protein or is pharmaceutically active in its own, e.g., it has one or more pharmaceutical activities such as those described for pharmaceutically active proteins. For example, the RNA may be one or more strands of RNA interference (RNAi). Such agents include short interfering RNAs (siRNAs), or short hairpin RNAs (shRNAs), or precursor of a siRNA or microRNA-like RNA, targeted to a target transcript, e.g., a transcript of an endogenous disease-related transcript of a subject.

A “pharmaceutically active peptide or protein” has a positive or advantageous effect on the condition or disease state of a subject when administered to the subject in a therapeutically effective amount. Preferably, a pharmaceutically active peptide or protein has curative or palliative properties and may be administered to ameliorate, relieve, alleviate, reverse, delay onset of or lessen the severity of one or more symptoms of a disease or disorder. A pharmaceutically active peptide or protein may have prophylactic properties and may be used to delay the onset of a disease or to lessen the severity of such disease or pathological condition. The term “pharmaceutically active peptide or protein” includes entire proteins or polypeptides, and can also refer to pharmaceutically active fragments thereof. It can also include pharmaceutically active analogs of a peptide or protein. The term “pharmaceutically active peptide or protein” includes peptides and proteins that are antigens, i.e., administration of the peptide or protein to a subject elicits an immune response in a subject which may be therapeutic or partially or fully protective.

Examples of pharmaceutically active proteins include, but are not limited to, cytokines and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, immunoglobulins, soluble major histocompatibility complex antigens, immunologically active antigens such as bacterial, parasitic, or viral antigens, allergens, autoantigens, antibodies), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Wilebrand factor, antithrombin III, glucocerebrosidase, erythropoietin granulocyte colony stimulating factor (GCSF) or modified Factor VIII, anticoagulants and the like.

In one embodiment, the RNA used in the present invention encodes a peptide or protein comprising an immunogen, antigen or antigen peptide. In one embodiment, the peptide or protein is processed after expression to provide said immunogen, antigen or antigen peptide. In another embodiment, the peptide or protein itself is the immunogen, antigen or antigen peptide. Cells expressing such peptide or protein comprising an immunogen, antigen or antigen peptide can be used, for example, in immunotherapy to elicit an immune response against the immunogen, antigen or antigen peptide in a patient.

The term “antigen” relates to an agent comprising an epitope against which an immune response is to be generated. The term “antigen” includes in particular proteins and peptides. The term “antigen” also includes agents, which become antigenic—and sensitizing—only through transformation (e.g. intermediately in the molecule or by completion with body protein). An antigen is preferably presentable by cells of the immune system such as antigen presenting cells like dendritic cells or macrophages. In addition, an antigen or a processing product thereof is preferably recognizable by a T or B cell receptor, or by an immunoglobulin molecule such as an antibody. In a preferred embodiment, the antigen is a disease-associated antigen, such as a tumor antigen, a viral antigen, or a bacterial antigen. In a preferred embodiment, the antigen comprises a tumor antigen or a portion thereof. In this embodiment, the compositions described herein may be useful in treating cancer or cancer metastasis.

The term “tumor antigen” refers to a constituent of cancer cells which may be derived from the cytoplasm, the cell surface and the cell nucleus. In particular, it refers to those antigens which are produced, preferably in large quantity, intracellularly or as surface antigens on tumor cells. Examples for tumor antigens include HER2, EGFR, VEGF, CAMPATH1-antigen, CD22, CA-125, HLA-DR, Hodgkin-lymphoma or mucin-1, but are not limited thereto.

The term “viral antigen” refers to any viral component having antigenic properties, i.e. being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “disease-associated antigen” is used in it broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen may therefore be used for therapeutic purposes. Disease-associated antigens are preferably associated with infection by microbes, typically microbial antigens, or associated with cancer, typically tumors.

“A portion or fragment of an antigen” or “an antigen peptide” according to the invention preferably is an oligopeptide or polypeptide comprising an amino acid sequence substantially corresponding to the amino acid sequence of a fragment or peptide of an antigen. An antigen peptide may be of any length.

Preferably, an antigen peptide is capable of stimulating an immune response, preferably a cellular response against the antigen or cells characterized by expression of the antigen and preferably by presentation of the antigen. Preferably, antigen peptides according to the invention are MHC class I and/or class II presented peptides or can be processed to produce MHC class I and/or class II presented peptides.

The term “in vivo” relates to the situation in a subject.

The terms “subject” and “individual” are used interchangeably and relate to mammals. For example, mammals in the context of the present invention are humans, non-human primates, domesticated animals such as dogs, cats, sheep, cattle, goats, pigs, horses etc., laboratory animals such as mice, rats, rabbits, guinea pigs, etc. as well as animals in captivity such as animals of zoos. The term “animal” as used herein also includes humans. The term “subject” may also include a patient, i.e., an animal, preferably a human having a disease.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “disease involving an antigen” refers to any disease which implicates an antigen, e.g. a disease which is characterized by the presence of an antigen. The disease involving an antigen can be an infectious disease, an autoimmune disease, or a cancer disease or simply cancer. As mentioned above, the antigen may be a disease-associated antigen, such as a tumor-associated antigen, a viral antigen, or a bacterial antigen.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

The term “autoimmune disease” refers to any disease in which the body produces an immunogenic (i.e. immune system) response to some constituent of its own tissue. In other words, the immune system loses its ability to recognize some tissue or system within the body as self and targets and attacks it as if it were foreign. Autoimmune diseases can be classified into those in which predominantly one organ is affected (e.g. hemolytic anemia and anti-immune thyroiditis), and those in which the autoimmune disease process is diffused through many tissues (e.g. systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the invention also comprises cancer metastases.

The term “treatment” or “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “protect”, “prevent”, “prophylactic”, “preventive”, or “protective” relate to the prevention and/or treatment of the occurrence and/or the propagation of a disease, e.g. tumor, in an individual. For example, a prophylactic administration of an immunotherapy, e.g. by administering the pharmaceutical composition described herein, can protect the receiving individual from the development of a tumor. For example, a therapeutic administration of an immunotherapy, e.g. by administering the pharmaceutical composition of the present invention, can stop the development of a disease, e.g. lead to the inhibition of the progress/growth of a tumor. This comprises the deceleration of the progress/growth of the tumor, in particular a disruption of the progression of the tumor, which preferably leads to elimination of the tumor. A therapeutic administration of an immunotherapy may protect the individual, for example, from the dissemination or metastasis of existing tumors.

The term “immunotherapy” relates to a treatment preferably involving a specific immune reaction and/or immune effector function(s).

The term “immunization” or “vaccination” describes the process of treating a subject for therapeutic or prophylactic reasons.

The pharmaceutical compositions described herein are preferably sterile and contain an effective amount of the RNA to be administered and optionally of further agents to generate the desired reaction or the desired effect.

For example, the pharmaceutical composition of the invention may be administered together with supplementing immunity-enhancing substances such as one or more adjuvants and may comprise one or more immunity-enhancing substances to further increase its effectiveness, preferably to achieve a synergistic effect of immunostimulation. The term “adjuvant” relates to compounds which prolongs or enhances or accelerates an immune response. Various mechanisms are possible in this respect, depending on the various types of adjuvants. For example, compounds which allow the maturation of the DC, e.g. lipopolysaccharides or CD40 ligand, form a first class of suitable adjuvants. Generally, any agent which influences the immune system of the type of a “danger signal” (LPS, GP96, dsRNA etc.) or cytokines, such as GM-CFS, can be used as an adjuvant which enables an immune response to be intensified and/or influenced in a controlled manner. CpG oligodeoxynucleotides can optionally also be used in this context, although their side effects which occur under certain circumstances, as explained above, are to be considered. Particularly preferred adjuvants are cytokines, such as monokines, lymphokines, interleukins or chemokines, e.g. IL-1, IL-2, IL-3, IL-4, IL-5, IL-6, IL-7, IL-8, IL-9, IL-10, IL-12, INFa, INF-γ, GM-CSF, LT-α, or growth factors, e.g. hGH. Further known adjuvants are aluminium hydroxide, Freund's adjuvant or oil such as Montanide®, most preferred Montanide® ISA51. Lipopeptides, such as Pam3Cys, are also suitable for use as adjuvants in the pharmaceutical composition of the present invention.

Pharmaceutical compositions are usually provided in a uniform dosage form and may be prepared in a manner known per se. The pharmaceutical composition of the invention may e.g. be in the form of a solution or suspension.

The pharmaceutical composition of the invention may comprise salts, buffer substances, preservatives, carriers, diluents and/or excipients all of which are preferably pharmaceutically acceptable. The term “pharmaceutically acceptable” refers to the non-toxicity of a material which does not interact with the action of the active component of the pharmaceutical composition.

Salts which are not pharmaceutically acceptable may used for preparing pharmaceutically acceptable salts and are included in the invention. Pharmaceutically acceptable salts of this kind comprise in a non limiting way those prepared from the following acids: hydrochloric, hydrobromic, sulfuric, nitric, phosphoric, maleic, acetic, salicylic, citric, formic, malonic, succinic acids, and the like. Pharmaceutically acceptable salts may also be prepared as alkali metal salts or alkaline earth metal salts, such as sodium salts, potassium salts or calcium salts.

Suitable buffer substances for use in the pharmaceutical composition of the invention include acetic acid in a salt, citric acid in a salt, boric acid in a salt and phosphoric acid in a salt.

Suitable preservatives for use in the pharmaceutical composition of the invention include benzalkonium chloride, chlorobutanol, paraben and thimerosal.

An injectable formulation may comprise a pharmaceutically acceptable excipient such as Ringer Lactate.

The term “carrier” refers to an organic or inorganic component, of a natural or synthetic nature, in which the active component is combined in order to facilitate, enhance or enable application. According to the invention, the term “carrier” also includes one or more compatible solid or liquid fillers, diluents or encapsulating substances, which are suitable for administration to a patient.

Possible carrier substances for parenteral administration are e.g. sterile water, Ringer, Ringer lactate, sterile sodium chloride solution, polyalkylene glycols, hydrogenated naphthalenes and, in particular, biocompatible lactide polymers, lactide/glycolide copolymers or polyoxyethylene/polyoxy-propylene copolymers.

The term “excipient” when used herein is intended to indicate all substances which may be present in a pharmaceutical composition of the present invention and which are not active ingredients such as, e.g., carriers, binders, lubricants, thickeners, surface active agents, preservatives, emulsifiers, buffers, flavoring agents, or colorants.

The agents and compositions described herein may be administered via any conventional route, such as by parenteral administration including by injection or infusion. Administration is preferably parenterally, in particular subcutaneously, intradermally or intramuscularly, more preferably intramuscularly.

The term “injection” may be understood as a generic term covering “injection” and “infusion”, but may also be understood as a specific method of administration, which is usually performed manually and in a short period of time. On the contrary, the term “infusion” may be understood as a method of administration wherein the fluid is continuously applied to the patient, usually by means of a specific feeding device. It should be noted that the injection or infusion may be carried out, among other things, by intradermal, subcutaneous, intramuscular, intravenous, intraosseous, intraperitoneal, intrathecal, epidural, intracardiac, intraarticular, intracavernous, or intravitreal administration.

“Intramuscular injection” is the injection of a substance directly into a muscle. Intramuscular injections are often given in the deltoid muscle of the arm, the vastus lateralis muscle of the leg, and the ventrogluteal and dorsogluteal muscles of the buttocks.

Compositions suitable for parenteral administration usually comprise a sterile aqueous or nonaqueous preparation of the active compound, which is preferably isotonic to the blood of the recipient. Examples of compatible carriers and solvents are Ringer solution and isotonic sodium chloride solution. In addition, usually sterile, fixed oils are used as solution or suspension medium.

The agents and compositions described herein are administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition.

An effective amount of an agent or composition described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the agents described herein may depend on various of such parameters. In the case that a reaction in a patient is insufficient with an initial dose, higher doses (or effectively higher doses achieved by a different, more localized route of administration) may be used.

The term “cannula” includes any tube or tube-shaped element or elongated element or cylindrical element such as a hollow needle or a flexible tube or hose element, which comprises a lumen for receiving a fluid and for supplying the fluid into a target area such as a target tissue of a patient. The cannula may be provided of any material which is in particular suitable for medical applications.

The term “fluid” means a composition that is in liquid form. Preferably, the term relates to a liquid pharmaceutical composition comprising one or more pharmaceuticals, in particular RNA, together with one or more excipients.

The term “target tissue” describes the part of an organ or tissue of a patient, which is intended for being treated with a fluid. The term “predetermined area of the target tissue” describes the part of the target tissue which is to be covered with a fluid. Although the present invention is in particular directed to the treatment of human or animal muscle tissue, the present invention is also applicable in connection with any other kind of tissue or in connection with any kind of cavities, like body cavities.

The drawings are not necessarily to scale. In certain instances, details that are not necessary for an understanding of the embodiment or that render other details difficult to perceive may have been omitted.

The same or equally acting components are provided with the same reference signs.

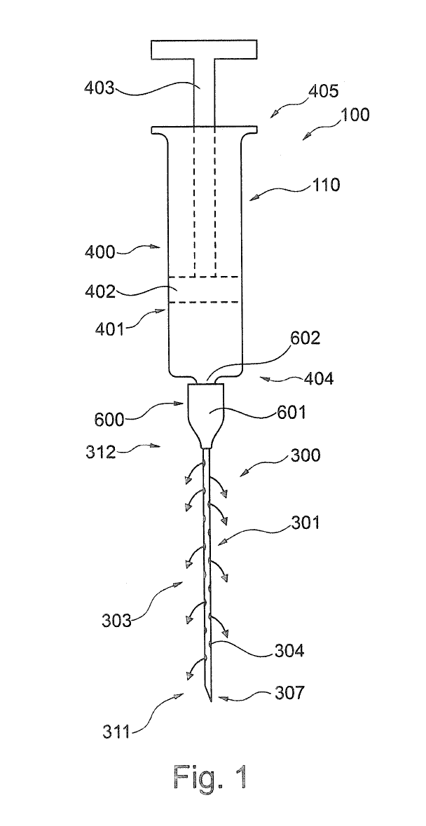

FIG. 1 shows a schematic side view of an injection or infusion device 100, in particular of an injection device, that is an injection syringe device, according to an embodiment of the present invention. The device 100 comprises a feeding device 400 for supplying a fluid 800 to a cannula 300, that is to a lumen 302 of the cannula 300. The feeding device 400 includes a tube-shaped or cylindrical element 401 and a piston element 402 sealingly slidable within the tube-shaped element 401 via a piston rod 403. The piston rod 403 protrudes from a proximal end 405 of the tube-shaped element 401 and thus, allows for moving the piston rod to supply the fluid to the cannula 300. Only the piston element 402 and the piston rod 403 are entirely depicted, thereby using dashed lines for those parts which are, in fact, not visible.

The injection device 100 further comprises the cannula 300, which is attached to the tube-shaped element 401, wherein a distal end 404 of the tube-shaped element 401 is connected with a proximal end 312 of the cannula 300.

The cannula 300 comprises a sidewall 301 which defines the lumen 302 for receiving the fluid 800 from the tube-shaped element 401, that is the feeding device 400. As can be taken from the Figure, the sidewall 301 comprises a plurality of openings 303 for supplying the fluid 800 into a target tissue 700 of a patient, wherein each opening 304 is in fluid communication with the lumen 302 of the cannula 300.

Conventional cannulas 30 (see FIG. 3) are configured to supply a fluid via an opening 36 formed at a tip 37 of the cannula 30 into a target tissue. The fluid is thus applied to the tissue surrounding the tip 37 or adjacent the tip so as to solely form one depot 81 of the fluid within the tissue. The depot 81 is the starting point for distributing the fluid, e.g. the active substance of a drug, within a predetermined area of the target tissue. As already indicated above, the depot thus formed is not sufficient to cover the predetermined area of the target tissue with the fluid, that is the active substance, since in most of the cases, the fluid cannot overcome the distance to peripheral areas of the predetermined area.

Due to the large number of openings 303 in the cannula 300 in accordance with the invention, the fluid 800 is applicable into a wide area within the target tissue 700, such as a muscle, thereby avoiding the accumulation of fluid within only one depot. That is, the plurality of openings 303 allows for forming a plurality of smaller depots 801 of the fluid 800 within a predetermined area 701 of the target tissue 700, instead of forming merely one large depot. The efficacy of the fluid, in particular of the drug, may therefore be increased, since the fluid and thus the active substance may cover even peripheral areas of the predetermined area 701. In other words: The plurality of depots 801 forms a plurality of starting points for distributing the fluid 800, e.g. the active substance of a drug, within the target tissue 700.

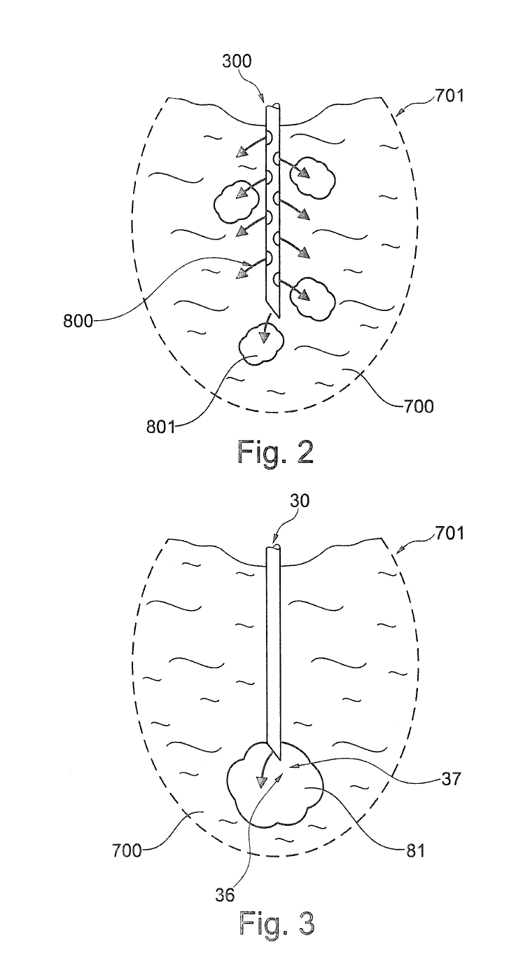

FIG. 2 shows a part of a cannula 300 according to an embodiment of the present invention, positioned in a target tissue 700 of a patient. FIG. 3 shows a part of a conventional cannula 30, also positioned in a target tissue 700 of a patient. The arrows in FIG. 2 (see also FIGS. 1 and 4 to 8) demonstrate the supply of the fluid 800 within the lumen 302 of the cannula 300 into the target tissue 700 via the plurality of the openings 303, thereby forming a plurality of depots 801 in a wide area of the target tissue 700. As can be taken from FIG. 3, due to the single opening of the cannula 30 at the tip 37 of the cannula 30, only one depot 81 is formed in the target tissue, close to the tip of the cannula. The single depot 81 does not allow for an even or uniform distribution of the fluid within the predetermined area of the target tissue.

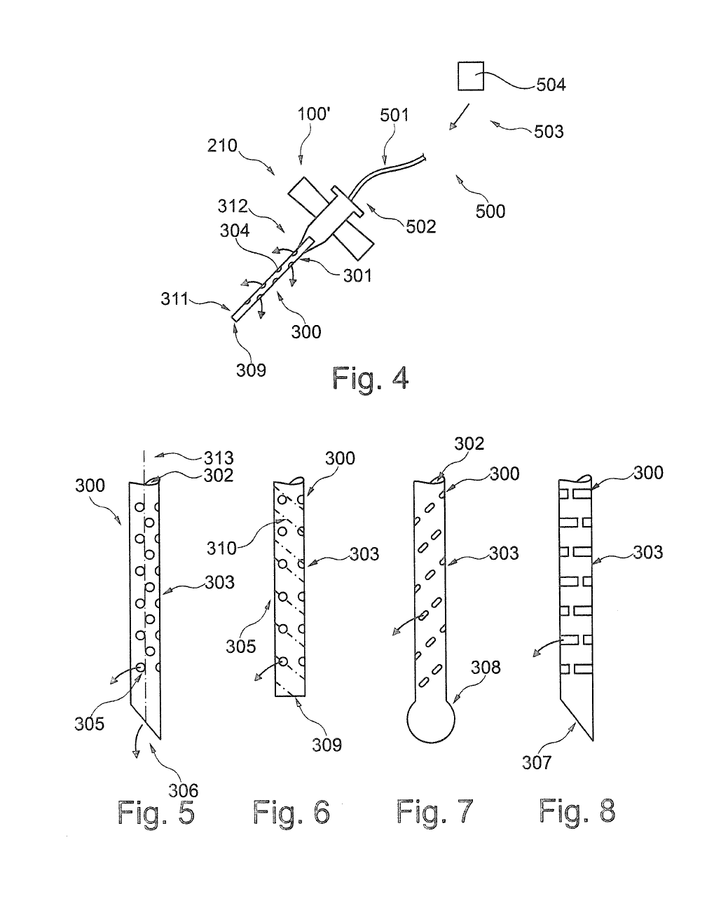

FIG. 4 shows a schematic side view of an injection or infusion device 200, in particular of an infusion device, according to an embodiment of the present invention. The infusion device 200 is provided as an indwelling or permanent catheter device comprising a feeding device 500 for supplying a fluid 800 to a cannula 300, that is to a lumen 302 of the cannula 300. The feeding device 500 comprises a connection tube or hose 501 and a reservoir 504 containing the fluid to be supplied into the target tissue of a patient, wherein the connection tube or hose 501 is configured to connect the reservoir 504 with the cannula 300. The reservoir 504 is for example provided as an infusion bag.

The cannula 300 is for example formed of a flexible material, such as polytetrafluoroethylene, polyurethane, polyamide or silicone and is preferably configured to receive a guiding mandrel or a guiding needle (not shown). The mandrel may be necessary for placing the cannula 300 into the target tissue 700, e.g. into a vein, and for compensating the flexibility of the cannula 300. Also rigid cannulas may be used for the infusion device.

The cannula 300 is attached to the connection tube 501, wherein a distal end 502 of the connection tube is connected with a proximal end 312 of the cannula 300. The proximal end 503 of the connection tube 501 is connected with the reservoir 504.

As can be taken from the Figure, the cannula 300 comprises a sidewall 301 or a lateral surface defining a lumen 302 (see e.g. FIG. 7) for receiving the fluid 800 from the reservoir 504. Similar to the embodiment described with FIG. 1, the sidewall 301 comprises a plurality of openings 303 for supplying the fluid 800 into the target tissue 700 of the patient, wherein each opening 304 is in fluid communication with the lumen 302 of the cannula 300.

The cannula 300 and the feeding device 400, 500 of the injection or infusion device 100, 200 may be provided as an integral component, that is in one piece, or the components may be provided as single elements which are connectable or attachable by means of a connection device 600, for example a female element or component 601 provided at the cannula 300 and a male element or component 602 provided at the feeding device 400, 500 or vice versa. The components of the injections or infusion device may be provided as disposable elements or components or are configured to be reusable.

As already described with the embodiment of FIG. 1, the plurality of openings 303 allows for applying the fluid into a wide area within the target tissue 700, thereby avoiding the accumulation of fluid within only one depot.

FIGS. 5 to 8 show several embodiments of the cannula 300. The cannula of FIG. 5 comprises a plurality of rows 305 of openings which extend from the proximal end 312 of the cannula 300 to the distal end 311, along a direction of extension of the cannula, that is along a longitudinal axis 313. The rows 305 extend off-set to one another in order to take as much advantage as possible of the lateral surface or sidewall 301, that is to provide as many opening as possible. FIG. 6 depicts rows 305 with openings provided at the same height on the sidewall 301, in respect of the direction of extension 313 of the cannula 300.

A helical arrangement of the plurality of openings 303 is shown with FIG. 7; FIG. 8 shows a grid-shaped structure which forms the plurality of openings 303. The opening may comprise a circular, oval and/or a slit-shaped structure. The cannula 300 may also comprise openings of different shape.

The cannulas 300 shown with FIGS. 5 and 8 comprise a tip 307 which is configured to penetrate the tissue of the patient in order to position the cannula 300 within the desired target tissue 700. That is, the cannulas comprise a tissue penetrating tip 307. FIG. 7 shows a buttoned cannula with a blunt distal end, that is with a button tip 308. Such cannulas may be used for example within an existing channel or opening or with a catheter which guides the cannula to the target tissue. The tips may comprise an additional opening 306, see for example FIG. 2 or 5, or may be closed. The additional opening at the tip is indicated with arrows in FIG. 2 or 5. The cannula of FIG. 4 is provided with a flat end tip 309, since a guiding mandrel comprising a tissue penetrating tip (not shown) allows for positioning the cannula within the target tissue. The flat end tip guarantees that no undesired penetration of the target tissue accidentally occurs.

The cannula 300 of FIG. 6 comprises a coating 310, for example an antiseptic and/or blood-repellant drug and/or a substance, for improving, among other things, the sliding characteristics of the cannula 300 within the tissue.

The arrows pointing away from the openings demonstrate the supply of the fluid into the tissue. For the purpose of improved clarity, not all of the openings are provided with a corresponding arrow, although all openings are used for supplying fluid into the tissue.

The described cannulas may be used with any type of injection or infusion devices, in particular in the medical and veterinary field. However, the cannulas may also be used in other fields, such as laboratory areas.

### EXAMPLES

### Example 1

### Distribution of lacZ-GFP-Luc Replicon RNA after Four Injections Per Muscle

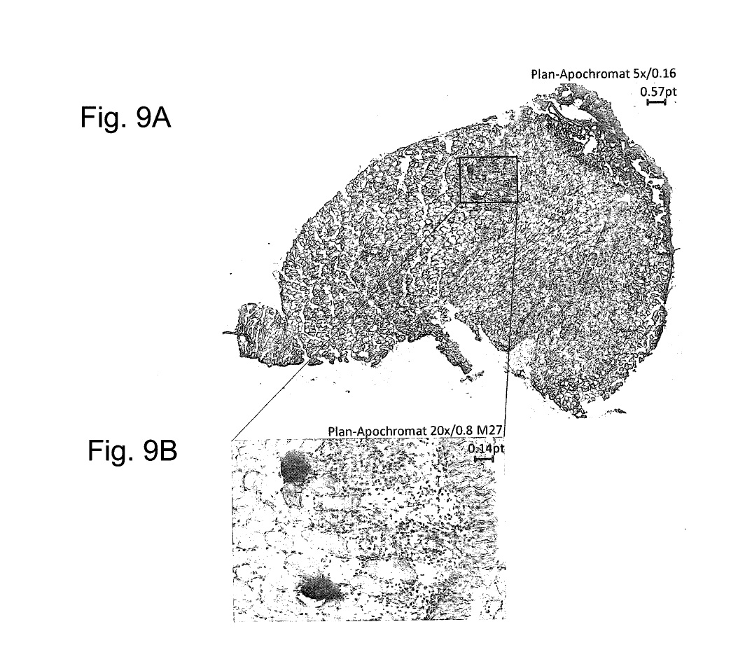

On day four after serially injecting (four times) lacZ-GFP-luc Replicon RNA (10 μg in 20 μl PBS) into right musculus tibialis posterior muscles of balb/c mice muscle tissues were prepared and immediately snap frozen. For determination of beta-galactosidase-positive cells, 12 μm thick cross-sections of the fresh frozen tissues were cut using a Leica cryotome (Leica, CM1950). Cells expressing β-galactosidase were detected following the manufacturer's protocol of the β-Gal Staining Kit (Life Technologies). In short, sections were fixed on glass slides and incubated at 37° C. for 90 min in β-Gal Staining solution. LacZ expressing cells catalyze the hydrolysis of beta-galactosides into its blue product indicating a successful penetration of a respective muscle cell. The staining procedure was followed by 2 min counterstaining with haematoxylin (Hamalaunlosung sauer nach Mayer, Carl Roth), 5 min bluing in tap water, and subsequent dehydration using an ascending alcohol row and finally Xylol and mounting with X-TRA KITT medite. Analysis and documentation were performed using either the AxiolmagerM2 (Zeiss) or the Axio Scan (Zeiss). The results in FIG. 9 show that per injection using conventional (one outlet port) insulin syringes with 8 mm and gauge 31 G needles only single muscle cells can be marked.

### LIST OF REFERENCE SIGNS

100 injection or infusion device: injection device, syringe device
200 injection or infusion device: infusion device
300 cannula
301 sidewall, lateral surface
302 lumen
303 plurality of openings
304 opening
305 row of openings
306 opening of tip
307 tissue penetrating tip
308 button tip
309 flat end tip
310 coating
311 distal end of cannula
312 proximal end of cannula
313 longitudinal axis, direction of extension
400, 500 feeding device
401 tube-shaped element, cylindrical element
402 piston element
403 piston rod
404 distal end of tube-shaped element
405 proximal end of tube-shaped element
501 connection tube or hose
502 distal end of connection tube
503 proximal end of connection tube
504 reservoir
600 connection device
601 female element, connection element
602 male element
700 target tissue
701 predetermined area of the target tissue
800 fluid
801 depot
30 conventional cannula
36 opening of tip
37 tip
81 depot

1. A cannula (300) with a distal end (311) and a proximal end (312), comprising:
a sidewall (301) defining a lumen (302) for receiving a fluid (800) from a feeding device (400, 500),
the sidewall (301) comprising a plurality of openings (303) for supplying the fluid (800) into a target tissue (700) of a patient, wherein each opening (304) is in fluid communication with the lumen (302) of the cannula (300).

2. The cannula according to claim 1,
wherein the cannula (300) is an injection or infusion cannula.

3. The cannula according to claim 1 or claim 2,
wherein the cannula (300) comprises a tissue penetrating tip (307) on the distal end (311).

4. The cannula according to any one of claims 1 to 3, in particular according to claim 1 or claim 2,
wherein the cannula (300) is a buttoned cannula comprising a button tip (308) on the distal end (311).

5. The cannula according to any one of claims 1 to 4,
wherein the plurality of openings (303) is evenly distributed on the sidewall (301) and/or entirely covers the sidewall (301).

6. The cannula according to any one of claims 1 to 5,
wherein the plurality of openings (303) form at least one row (305) which extends along a longitudinal axis (313) of the cannula (300) from the proximal end (312) of the cannula (300) to the distal end (311).

7. The cannula according to any one of claims 1 to 6,
wherein the openings form a plurality of rows.

8. The cannula according to any one of claims 1 to 7,
wherein the one or more rows (305) extend in a helical manner from the proximal end (312) of the cannula (300) to the distal end (311).

9. The cannula according to any one of claims 1 to 8,
wherein the openings comprise circular, oval and/or a slit-shaped structure.

10. The cannula according to any one of claims 1 to 9,
wherein the sidewall (301) comprises a grid-shaped structure which forms the openings.

11. The cannula according to any one of claims 1 to 10,
wherein the tissue penetrating tip (307) and/or the button tip (308) comprise a tip opening (306).

12. The cannula according to any one of claims 1 to 11,
wherein the cannula (300) is attached or is configured to be attachable to the feeding device (400, 500) of an injection or infusion device (100, 200).

13. The cannula according to any one of claims 1 to 12,
wherein the cannula (300) comprises a connection element (601), preferably a funnel-shaped connection element, for attaching the cannula (300) to the feeding device.

14. The cannula according to any one of claims 1 to 13,
wherein the cannula (300) comprises a length in the range of 10 to 120 mm, preferably of 40, 50, 60, 70 or 80 mm.

15. The cannula according to any one of claims 1 to 14,
wherein the cannula (300) comprises a diameter in the range of 0.5 to 1.2 mm, preferably of 0.6, 0.9 or 1.0 mm.

16. The cannula according to any one of claims 1 to 15,
wherein the material of the cannula (300) is of stainless steel, polytetrafluoroethylene, polyurethane, polyamide or silicone.

17. The cannula according to any one of claims 1 to 16,
wherein the cannula (300) is coated (310) with a hydrogel and/or a drug.

18. The cannula according to any one of claims 1 to 17,
wherein the cannula (300) is configured to receive a guiding mandrel or a guiding needle.

19. The cannula according to any one of claims 1 to 18,
wherein the cannula (300) is formed as a disposable component.

20. An injection or infusion device (100, 200) comprising:
a cannula (300) according to any one of claims 1 to 19,
a feeding device (400, 500) for supplying the fluid (800) to the cannula (300),
wherein the cannula (300) is attached or is configured to be attachable to the feeding device (400, 500).

21. The injection or infusion device according to claim 20,
wherein the feeding device (400) comprises:
a tube-shaped or cylindrical element (401) comprising a distal end (404) and a proximal end (405), the tube-shaped or cylindrical element (401) for receiving the fluid (800) to be supplied into the target tissue (700) of a patient, and
a piston element (402) sealingly slidable within the tube-shaped or cylindrical element (401), and
a piston rod (403) for moving the piston element (402) within the tube-shaped or cylindrical element (401).

22. The injection or infusion device according to claim 20,
wherein the feeding device (500) comprises:
a reservoir (504) containing the fluid (800) to be supplied into the target tissue (700) of a patient, and
a connection tube or hose (501) for connecting the reservoir (504) with the cannula (300).

23. The injection or infusion device according to any one of claims 20 to 22,
wherein the cannula (300) is attached or is configured to be attachable to the feeding device (400, 500) by means of a connection device (600).

24. The injection or infusion device according to any one of claims 20 to 23,
wherein the feeding device (400, 500) and the cannula (500) are formed as an integral component or in one piece.

25. The cannula according to any one of claims 1 to 19 or the injection or infusion device according to any one of claims 20 to 24, wherein the fluid is a pharmaceutical composition comprising RNA.

26. A method for delivering RNA to cells of a subject, comprising administering to the subject a fluid comprising the RNA using the cannula according to any one of claim 1 to 19 or 25 or the injection or infusion device according to any one of claims 20 to 25.

27. A method for expressing RNA in cells of a target organ or tissue comprising introducing into the target organ or tissue a fluid comprising the RNA using the cannula according to any one of claim 1 to 19 or 25 or the injection or infusion device according to any one of claims 20 to 25.

28. The method of claim 27, wherein the RNA once introduced into the target organ or tissue is taken up by cells of the target organ or tissue.

29. A method of treating or preventing a disease in a patient comprising administering to the patient a fluid comprising RNA using the cannula according to any one of claim 1 to 19 or 25 or the injection or infusion device according to any one of claims 20 to 25.
